(19) United States
(12) Patent Application Publication
(10) Pub. No.: US 2006/0246537 A1
(43) Pub. Date: Nov. 2, 2006

(54) Multi-step process for the manufacture of therapeutic protein

(76) Inventors: Lauri Laurene Jenkins, Newark, DE (US); Jay Irving Kennedy, Circleville, OH (US); Victor Sisto Lusvardi, Chadds Ford, PA (US); Toni T. Butler, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/412,329

(22) Filed: Apr. 27, 2006

Related U.S. Application Data
(60) Provisional application No. 60/676,528, filed on Apr. 29, 2005.

(51) Int. Cl. C12P 21/06; C07K 14/47; A61M 31/00; A61K 38/16

(52) U.S. Cl. 435/68.1; 435/69.1; 514/12; 530/350; 604/500

(57) ABSTRACT

A process for the production of therapeutic protein is provided, which includes the steps of preparing nutrient medium for culturing of cells to express the protein, culturing the cells in the presence of the nutrient medium to express the protein, preparing protein separation solution for isolating the protein, formulating the isolated protein, and storing the formulated protein, at least three of these steps being carried out in separate disposable containers made of flexible film, at least the interior of said surface of said container being made of fluoropolymer.

### BACKGROUND OF THE INVENTION

1. Field of the Invention

This invention relates to the manufacture of therapeutic protein and more particularly to the vessels in which the manufacture is carried out.

2. Description of Related Art

DNA technology involves multiple steps, including formation of the genetically altered cell line, fermenting or culturing the cell line to express the protein, including the preparation of the nutrient medium, purifying the protein, including the preparation of protein separation solutions, and formulating and storing the protein. The protein is subject to undesirable alteration and even denaturing by the presence of contaminants in any of the solutions containing the protein in one or more of the manufacturing steps. For commercial operation, the vessels used in carrying out the steps in the process are primarily stainless steel, thought to be corrosion resistant and thus non-contaminating to the different media present in the manufacturing steps. With the use of stainless steel, however, the manufacturing line has to be shut down periodically for clean-in place operations (between production batches) and corrosion remediation. The stainless steel vessels show effects of corrosion, such as “rouging” or pitting of the interior surface of the vessel, which indicates that the vessel has contributed to contamination of the medium contained in the vessel. The clean-out process is costly, involving such steps as cleaning of the vessel, electro-polishing its interior surface, sterilizing the resultant surface, and validation that the re-furbished vessel can be returned to service. The loss of production and possibly loss of therapeutic protein that led to the shut down is also costly.

The problem remains on how to provide a non-contaminating surface for the vessels used in the manufacture of the therapeutic protein, so as to avoid the need for and expense of periodic shut down and clean out and prevent the loss of therapeutic protein from contamination.

### SUMMARY OF THE INVENTION

The present invention solves this problem by providing a new material of construction for vessels used in the manufacture of therapeutic protein, which vessels are non-contaminating and do not require cleaning. In greater detail, the problem is solved by the process of the present invention for the production of therapeutic protein, which comprises (a) preparing nutrient medium for fermenting or culturing of cells to express said protein. (b) fermenting or culturing of said cells in the presence of said nutrient medium to express said protein, (c) preparing protein separation solution for isolating said protein, (d) formulating the isolated protein, and (e) storing the formulated protein, at least three of the steps (a)-(e) being carried out in separate disposable containers made of flexible film, at least the interior of said surface of said containers being fluoropolymer. The flexibility of the film imparts flexibility to the container, which promotes the ability to package the container for installation into particular steps in the process, and removal upon completion of their life in the process step for replacement by another container made of the same film. The disposability eliminates the need for cleaning and validation of sterilization and reduces production downtime to container replacement. An additional benefit of the disposable containers in the manufacturing steps is the absence of cross-contamination, i.e. the carryover of contamination provided by one vessel to the vessel used in the succeeding steps in the production process.

In preferred embodiments, at least steps (a), (b), and (c) are carried out in said disposable containers and more preferably, all said steps (a)-(e) are carried out in said disposable containers. In another preferred embodiment, each container is made entirely of the fluoropolymer.

The cell culturing occurring in the expression of a therapeutic protein, which is different from the cell line from which the protein is expressed, in accordance with the present invention differs from the cell culturing that involves only the growing of the original cells, without creating a different cellular product. The latter cell culturing is represented for example by the immunotherapy application for the disposable bags in U.S. Pat. No. 4,847,462. In contrast, the cell culturing occurring in the expression process of the present invention produces a non-living protein from the living, growing cell culture. This protein product is more susceptible to harm from organics contamination than the host cell culture. The host cell culture is a living organism and can therefore make some adjustment to counteract such contamination. The expressed protein, because it is not a living organism, cannot make this adjustment. Consequently, the likelihood of an organic (contamination) to organic (protein) reaction to adversely affect the protein is much greater. In addition, the therapeutic protein is a small fraction of the cell culture from which the protein is expressed. Therefore, an amount of organics contamination that might be small relative to the cell culture, will be large relative to the amount of therapeutic protein. Another difference from mere cell culturing is that the expression process is usually carried out in a succession of reactors of increasing volume, within each of which the cell culturing is conducted until optimum density is reached. The cell culture/expressed therapeutic protein medium is thus exposed to a plurality of bioreactor surfaces in advancing from one bioreactor to the next, thereby being subject to contamination by each reactor surface, instead of exposure to only one container surface as in the case of mere cell culturing, i.e. unaccompanied by expression of different product. The surprising resistance to extraction of organics from the fluoropolymer forming at least the interior surface of the container(s) (bioreactors) in which the expression process of the present invention is carried out enables the therapeutic protein to be preserved as formed for supply to the step(s) of recovering this protein from the biomass within which it was produced. This is especially surprising when the container is sterilized prior to use as the bioreactor by exposure to degradative ionizing radiation, such as by gamma radiation, as will be further described hereinafter.

In still another preferred embodiment, the above process includes providing each said containers as a package comprising a sealed overwrap containing said container, said container being sterilized within sealed overwrap, thereby retaining its sterilized condition until opening of said overwrap. The sterilization is preferably carried out by exposing each container to ionizing radiation through their respective sealed overwrap. In at least one of the process steps (a)-(d) described above, the water used to form the aqueous medium used in the process step is preferably highly purified water. More preferably, the highly purified water is used at least in steps (a) and (c) and possibly in steps (b) and (d). Typically, this water has been stored in a vessel of stainless steel so as to be available when needed in the protein manufacturing process. Unfortunately, the stainless steel provides some contamination to the highly purified water, which is then brought into the process step in which the water is used. This problem is solved by storing the water in a container of flexible film, at least the interior surface of the container being fluoropolymer. Preferably, this container is also disposable. This storage process can be used in combination with the protein manufacturing process described above or can be used independent thereof. The foregoing described highly purified water is commonly known as water for injection (WFI). WFI is defined in United States Pharmacopeia (USP) 1231 under Water for Pharmaceutical Purposes. In substance, WFI is highly purified water, the purity of which is designed to prevent microbial contamination and the formation of microbial endotoxins. WFI is also well known as being highly corrosive material, which provides a severe test of extractability of organics (organic compounds) from any polymer container. Resistance to extraction is determined by maintaining the copolymer container being tested and containing 250 ml of the WFI at 40° C. for 63 days, followed by analysis of the WFI for organics, that could only come from (by extraction from) the container copolymer. For the above-described container. No organics were found in the WFI present in the container under the above conditions. The detection limit for the analysis was 50 ppb. Further details on the analysis as part of the extraction test and the application of this test to other test liquids and other polymers are disclosed later herein.

### DETAILED DESCRIPTION OF THE INVENTION

Protein therapies are made by expression from the culturing of cells in a fermentation broth or cell culture from a cell line. Typically, the cell line is recombinant, i.e. one or more cells are genetically altered by combination with DNA from a different organism, and these recombinant cells are cloned to form a cell bank. The cloning of a cell produced by recombinant DNA is well known in the art. Aliquots are taken from this cell bank for fermenting or culturing, and the therapeutic protein is expressed during growth (propagation) of the cells in the fermentation or cell culture process. In the case of the cell line being recombinant, the resultant expressed protein is also recombinant. The expression of the protein (step (b) of the process) is typically carried out by inoculating a fermentation broth or cell culture medium with the aliquot of the cell line into a nutrient medium into which is bubbled oxygen and nitrogen and accompanied by mixing so that the cell culturing conditions within the medium is homologous and at a controlled temperature. The vessel in which this bioreaction is carried is called a bioreactor. Typically, the reaction in step (b) is replicated in a succession of at least two bioreactors in increasing volume, this succession being called an inoculum train, the increase in volume designed to establish the best condition for optimum cell growth (cell density) within each bioreactor and thus optimum expression of the therapeutic protein in the overall manufacturing process. When the optimum amount of cell culture is produced in one bioreactor, the fermentation broth or cell culture medium is transferred to a larger volume bioreactor, wherein optimum conditions are established to increase the expression (production) of the therapeutic protein. In each bioreactor, the nitrogen purges carbon dioxide from the nutrient medium, which is formed during the expression process, and optimum amounts of nutrient medium, oxygen, and carbon dioxide are maintained to provide optimum cell growth and therapeutic protein production. The optimum pH is also established, monitored and maintained. These conditions are well known in the art and are individualized for the particular cell line being fermented or cultured and the particular therapeutic protein being formed.

The preparation of nutrient media is also well known in the art and is individualized as just described. The function of the nutrient medium to make the cells of the particular cell line in the bioreactor grow and in the course of growing, express the desired protein. The nutrient medium is an aqueous solution typically including an energy source, usually one or more sugars, to stimulate cell growth, and will typically include additional ingredients, such as minerals, amino acids, and vitamins, to mimic natural biologic fluid stimulative of cell growth for the particular cell line. The nutrient medium will also include strong acid(s) or base(s) and buffers to define and control pH of the nutrient medium, and some or all of these ingredients and others in the nutrient medium corrode or otherwise extract contaminants from the stainless steel bioreactor surface in contact with the medium, both in the preparation and fermentation and cell culturing processes. Examples of ingredients in the nutrient medium include calcium chloride solution, glucose, lactalbumin hydrolysate, soy hydrolysate, glutamine, sodium pyruvate, and tryptose phosphate broth. Nutrient media are sometimes purchased from nutrient medium manufacturers and are sometimes prepared by the protein manufacturer, by mixing the nutrient medium ingredients with water in a container. In either case, this container can be used for preparation and storage of the nutrient medium, or separate containers can be used. Typically the nutrient medium is pumped through a sterilizing filter (microorganism size exclusion filter) into the bioreactor(s) for carrying out the expression process.

Preferably other agents that are used in the expression process are also prepared and/or storing in separate containers made of flexible film, at least the interior surface of each said container being fluoropolymer. Such other agents include activator (induction agent), buffer, acid and/or base.

Downstream from the process of making a therapeutic protein by expression from cell culture, possibly from a recombinant cell line, the protein must be purified to separate it from undesirable materials including undesirable protein (protein contaminant) present in the fermentation broth or cell culture medium in which the therapeutic protein is made. After centrifugation to remove excess water, the purification typically involves filtration and/or chromatography, assisted by the addition of one or more protein separation solutions in the chromatography separation process. The protein separation solutions include buffered aqueous solutions or highly concentrated aqueous salt solutions, or combinations thereof of varying pH, depending on the adsorption matrix being used in the protein separation process being carried out. Protein separation processes include gel/filtration/size exclusion chromatography, ion exchange chromatography, hydrophobic interaction chromatography, affinity chromatography. In size exclusion chromatography, the adsorption of the matrix is in effect a partitioning between molecules of different hydrodynamic radius as the protein solution passes through the matrix. Buffers such as phosphate buffered saline solution are typically added to the protein solution to aid in the partitioning process. In ion exchange chromatography, the electrical charge on the adsorption matrix attracts the oppositely charged therapeutic protein or contaminant, as the case may be, whereby the unattracted protein or contaminant passes through the matrix, thereby separating the protein and contaminant, one from the other. When the therapeutic protein is the attracted (bound) material and the matrix is anion exchange, buffers having a pH of 7-10 are used, to which sodium chloride is added to release the bound protein from the matrix (elution). When the matrix is cation exchange, the elution buffer will typically have a pH of 4 to 7 and sodium chloride will be added to the buffer to obtain the elution of the adsorbed protein. In hydrophobic interaction chromatography, separation is based on selective hydrophobic interactions of ingredients in the therapeutic protein solution. For example, selective attractive of the therapeutic protein to the matrix can be obtained pre-washing the matrix with 2M ammonium sulfate, and elution of the bound protein can be obtained by washing the matrix with lower ionic strength buffer. In affinity chromatography, a ligand is used in the matrix to bind to the specific ingredient desired. Binding can be done using a neutral pH buffered solution and elution can be done using a buffer solution having a pH of 3. Examples of elution buffers include 0.1 M glycine-NaOH, pH 10, 0.1 M glycine-HCl, pH 3, and high salt buffers such as those having at least 3 moles of MgCl₂, KCl, or KI. Buffer solutions for all these separation processes are available from various suppliers. Preparation of these aqueous solution(s) is step (c) of the process of the present invention. These solutions are highly corrosive to the stainless steel mixing and storage tanks ordinarily used for their preparation. The corrosion problem is exacerbated by long periods of storage of the solutions so as to be available when needed. At this point, avoidance of contaminating the therapeutic protein is critical, because there is no additional purification step to remove the contamination and such contamination can cause changes to the protein, even denaturing it.

U.S. Patent publications 2004/0236083 and 2004/0242855 disclose the chromatographic separation of the desired protein from a protein solution that contains contaminant, typically a protein contaminant, by contacting the solution with an adsorptive matrix to adsorb either the therapeutic protein or the contaminant from the solution, thereby separating these proteins from one another. These publications disclose the use of a concentrated salt solution of low pH to contact the adsorptive matrix material prior to carrying out the separation process, this pre-contacting serving to aid in the separation achieved by the adsorptive matrix. These publications also disclose the use of concentrated salt solution of high pH to elute the adsorbed protein from the adsorptive matrix after carrying out the separation process. These are examples of the corrosive protein separation solutions that are prepared in step (c) for used in the protein separation process carried out as part of the purification of the therapeutic protein. These publications disclose the use of a fluoropolymer vessel such as a chromatography column, either made entirely of the fluoropolymer or lined with fluoropolymer, the liner being adhered to the vessel or column within which the separation is carried out. The advantage of this use of fluoropolymer instead of the usual material of construction, stainless steel. Is that the fluoropolymer does not contaminate the protein solution and thereby the therapeutic protein with metal as does the stainless steel vessel. These publications also disclose the periodic cleaning of the vessel by washing with caustic solution. While Publications 2004/0236083 and 0242855 address the problem of avoiding metallic contamination in the chromatography separation process, there remains the problem of the protein separation solutions not bringing contamination into the chromatograph separation process, particularly from the preparation of such solutions and especially from the storage of such solutions, wherein the solution remains in contact with the vessel interior surface for considerable time. The preparation and storage may be carried out in the same vessel, in which case, step (c) can include the storage of the prepared solution. The disposability of the container(s) used in step (c) as well as in the other process steps eliminates the need for cleaning such as the caustic cleaning discloses in the above patent publications. Such cleaning must ordinarily be followed by the additional step of verification that the vessel surface is not only clean, but is also free of microorganism, i.e. is sterile. The need for verification is also eliminated by the present invention.

Downstream from the protein separation process using the protein separation solution(s) of step (c) of the process, the purified therapeutic protein next has to be formulated so as to be deliverable to obtain the desired therapeutic result. The therapeutic protein is typically received from the purification process as an aqueous solution, such as in the aqueous solution used to elute the therapeutic protein selectively adsorbed in the chromatographic separation process. Formulation (step (d)) typically involves the addition of aqueous buffer to the protein solution since maintenance of pH may be important to the storage stability of the protein. Salts may also be added to improve solubility of the protein in the aqueous solution. Other excipients that may be added include stabilizers, antimicrobials, preservatives, surfactants, antioxidants, and isotonicity agents, to maintain the efficacy of the protein during storage (step (e)), whether at room temperature, chilled or cryopreserved. Thus, the formulation process includes the mixing together of the protein solution with buffer, possibly salt(s) and excipients. It is critical to be able to formulate the purified protein without the vessel within which the formulation process is carried out contaminating the formulation. Such contamination can diminish the efficacy of the protein, provided efficacy variation from batch-to-batch, and even denature the protein.

The formulation of therapeutic proteins and storage of the formulated protein are well-known to persons of ordinary skill in the art of manufacturing therapeutic proteins, including those derived from cell lines made by recombinant DNA. Since the therapeutic protein has already been purified prior to reaching the formulation process, any contaminant introduced by the vessel(s) within which the formulation and/or storage is not removed and therefore stays with the therapeutic protein, even into the fill and finish processes in which the protein is made ready for delivery to the patient. The formulated protein is generally very dilute in aqueous solution, whereby even small amounts of contaminant represent large amounts relative to the amount of protein present in the formulation. Apart from the relativity of amounts, small amounts of contaminant can have appreciable adverse effects on the protein, diminishing its efficacy, causing efficacy variations from batch-to-batch, and even destroying the efficacy of the protein.

The water used to form the aqueous media used in the foregoing-described process steps is preferably water for injection (WFI). This highly purified water satisfies the requirements for compendial water established by the USP (United States Pharmacopoeia) monographs for purified water USP or water for injection USP. Preferably the excipients and adjuvants added to the therapeutic protein solution in the formulation process are also prepared and/or stored prior to such addition in separate containers made of flexible film, at least the interior surface of each container being fluoropolymer

The present invention provides a container (vessel) applicable for use in any and all of the protein manufacturing process steps (a)-(e) described above and for storing the highly purified water, activator and acids, base and/or buffer, such container providing much improved resistance to contaminating the particular medium present in the vessel within which process step is carried out and thus, non-contaminating to the therapeutic protein, and as described above, improved economy and of operation.

The container used in the present invention is made of flexible film, the surface of which forming the interior surface of the container is fluoropolymer. The fluoropolymers used in the present invention are melt-fabricable, which means that they are sufficiently flowable in the molten state (heated above its melting temperature) that they can be fabricated by melt processing, preferable extrusion such as to form a film that is optically clear. Typically, the fluoropolymer by itself is melt-fabricable; in the case of polyvinyl fluoride, the fluoropolymer is mixed with solvent for extrusion, i.e. solvent-aided extrusion. The resultant film has sufficient strength so as to be useful. The melt flowability of the fluoropolymer can be described in terms of melt flow rate as measured in accordance with ASTM D-1238, and the fluoropolymers of the present invention preferably have a melt flow rate of at least 1 g/10 min, determined at the temperature which is standard for the particular fluoropolymer; see for example, ASTM D 2116a and ASTM D 3159-91a. Polytetrafluoroethylene (PTFE) is generally not melt processible, i.e. it does not flow at temperatures above the melting temperatures, whereby this polymer is not melt-fabricable. PTFE film is also not optically clear. Optical clarity is desired so that when the film is fabricated into a container, the interior of the container can be observed through the film wall of the container, enabling the observer to confirm that no visible contaminant or evidence of contamination such as the appearance of turbidity is present. Low molecular weight PTFE is available, called PTFE micropowder, the molecular weight being low enough that this polymer is flowable when molten, but because of the low molecular weight, the resultant molded article has no strength. The absence of strength is indicated by the brittleness of the article. If a film can be formed from the micropowder, it fractures upon flexing. In contrast, the melt-fabricable fluoropolymers used in the present invention can be formed into films that can be repeatedly flexed without fracture. This flexibility can be further characterized by an MIT flex life of at least 500 cycles, preferably at least 1000 cycles, and more preferably at least 2000 cycles, measured on 8 mil (0.2 mm) thick compression molded films that are quenched in cold water, using the standard MIT folding endurance tester described in ASTM D-2176F. The flexibility of the container enables it to collapse into a flattened shape. Flexibility can also confirmed by attempting to puncture the film from which the container is made, such as by following the procedure of ASTM F1342, with the result that prior to puncture, the stylus used in the puncture test deflects the film from its planar disposition in the test to the extent of at least about 5 times the thickness of the film being tested, and preferably at least 10 times the film thickness.

The preferred melt-fabricable fluoropolymers for use in the present invention comprise one or more repeat units selected from the group consisting of —CF₂—CF₂—, —CF₂—CF(CF₃)—, —CF₂—CH₂—, —CH₂—CHF— and —CH₂—CH₂—, these repeat units and combinations thereof being selected with the proviso that said fluoropolymer contains at least 35 wt % fluorine, preferably at least 50 wt % fluorine. Thus, although hydrocarbon units may be present in the carbon atom chain forming the polymer, there are sufficient fluorine-substituted carbon atoms in the polymer chain to provide the desired minimum amount of fluorine present, so that fluoropolymer exhibits chemical inertness. The fluoropolymer preferably also has a melting temperature of at least 150° C., preferably at least 200° C., and more preferably at least 240° C.

Examples of perfluoropolymers, i.e., wherein the monovalent atoms bonded to carbon atoms making up the polymer are all fluorine, except for the possibility of other atoms being present in end groups of the polymer chain, include copolymers of tetrafluoroethylene (TFE) with one or more perfluoroolefins having 3 to 8 carbon atoms, preferably hexafluoropropylene (HFP). The TFE/HFP copolymer can contain additional copolymerized perfluoromonomer such as perfluoro(alkyl vinyl ether), wherein the alkyl group contains 1 to 5 carbon atoms. Preferred such alkyl groups are perfluoro(methyl vinyl ether), perfluoro(ethyl vinyl ether) and perfluoro(propyl vinyl ether). Typically, the HFP content of the copolymer is about 7 to 17 wt %, more typically about 9 to 17 wt % (calculation: HFPI×3.2), and the additional comonomer when present constitutes about 0.2 to 3 wt %, based on the total weight of the copolymer. The TFE/HFP copolymers with and without additional copolymerized monomer is commonly known as FEP. Examples of hydrocarbon/fluorocarbon polymers (hereinafter “hydrofluoropolymers”) include vinylidene fluoride polymers (homopolymers and copolymers), typically called PVDF, copolymers of ethylene (E) with TFE, typically containing 40 to 60 mol % of each monomer, to total 100 mol %, and preferably containing additional copolymerized monomer such as perfluoroalkyl ethylene, preferably perfluorobutyl ethylene. These copolymers are commonly called ETFE. While ETFE is primarily composed of ethylene and tetrafluoroethylene repeat units making up the polymer chain, it is typical that additional units of from a different fluorinated monomer will also be present to provide the melt, appearance, and/or physical properties, such as to avoid high temperature brittleness, desired for the copolymer. Examples of additional monomers include perfluoroalkyl ethylene, such as perfluorobutyl ethylene, perfluoro(ethyl or propyl vinyl ether), hexafluoroisobutylene, and CH₂═CFR_{f} wherein R_{f} is C₂—C₁₀ fluoroalkyl, such as CH₂═CFC₅F₁₀H, hexafluoropropylene, and vinylidene fluoride. Typically, the additional monomer will be present in 0.1 to 10 mol % based on the total mols of tetrafluoroethylene and ethylene. Such copolymers are further described in U.S. Pat. Nos. 3,624,250, 4,123,602, 4,513,129, and 4,677,175. Additional hydrofluoropolymers include EFEP and the copolymer of TFE/HFP and vinylidene fluoride, commonly called THV. Films of these copolymers are all commercially available. Typically the film from which the container is made will have a thickness of about 2 to 10 mils (0.05 to 0.25 mm).

The fluoropolymer forms at least the inner surface of the container, i.e. the container may be formed from a film that is laminate in which the fluoropolymer layer faces the interior of the container. Preferably however, the fluoropolymer forms the entire thickness of the film, whereby the container is made entirely of the fluoropolymer. In either case, the bag will have a film thickness as stated in the preceding paragraph. The mono (single) layer film has the advantage of avoiding the need to laminate or otherwise bond the fluoropolymer layer of the laminate to the outer layer thereof. This has the further advantage in forming seams in the fabrication of the film into a container. The seam will involve heat bonding fluoropolymer to itself and edges of the film present in the seam in the interior of the container will be entirely fluoropolymer. The fluoropolymer layer or monolayer film as the case may be is non-adherent with respect to the nutrient medium, fermentation broth and cell culture medium, the expressed therapeutic protein, the protein separation solutions and the protein formulation, i.e. the ingredients in these media do not adhere to the fluoropolymer surface in contact with these media. Nor does the highly purified water adhere to the fluoropolymer surface. The film, whether a laminate or monolayer is preferably optically clear, so that the interior of the container made from the film can be observed through the film wall of the container, enabling the observer to confirm that no visible contaminant is present when the container is supplied in a package as will be explained hereinafter

The container can have any configuration and size desired for application in the particular step in the manufacture of therapeutic proteins or for the storage of purified water. For example, the container can be formed from two sheets of film heat sealed together along their edges to form an envelope. Alternatively, the container can be formed from sheets of film to form a container with distinct bottom and sides, either to form a round-sided container or one with distinct sides coming together at corners. Whatever the configuration, the container forms a vessel, within which a step in the protein manufacture can be carried out. The container can be open at the top (in use) or can be closed, except for a port of entry for the medium to be made or used. The port of entry can simply be a length of tubing heat sealed to the film forming the container. The entry port can be located elsewhere in the container and additional openings can be provided, such as equipped with tubing heat sealed to the film of the container, for such processing activities as discharge of the liquid contents from the container, feed of gas to the container, or multiple gases as in the case when the container is used as the bioreactor, wherein both oxygen and nitrogen are introduced to the fermentation broth/nutrient medium or cell culture/nutrient medium in the bag, and an additional port is provided to enable carbon dioxide to vent from the container. An additional port can be provided for the introduction of a mixing blade into the interior of the container. The tubing heat sealed to container(s) is preferably also made of fluoropolymer. Such tubing can be used to communicate liquid medium from one container to another in the manufacturing process, whereby the principal contact surfaces in the manufacturing process is all fluoropolymer. The same result is accomplished when the communication between container in the process is done by dumping the contents of one container into another where applicable in the process. Examples of bag configurations include those shown in U.S. Pat. Nos. 5,941,635, 6,071,005, 6,287,284, 6,432,698, 6,494,613, 6,453,683, and 6,684,646.

The interior volume of the container can be such as to accommodate either the research manufacture of the protein or the commercial manufacture thereof. Typically, the volume of the container will be at least 500 ml, but more typically, at least 1 liter, but sizes (volumes) of at least 10 liter, at least 50 liter, at least 100 liters, and at least 1000 liters, and even at least 10,000 liters are possible. Since the fluoropolymer film can be made in practically unlimited length, it is only necessary to cut this length into the lengths desired and fabricate these lengths together to form the container with the configuration and size desired. Small container sizes can be used unsupported, while a rigid support can be used for larger container sizes. The rigid support could be simply a base upon which the container rests or a rigid housing within which the bag is positioned so that both the bottom and side(s) of the container are supported. When the rigid support will be necessary will depend on the size of the container and its film thickness. The rigid support can be existing vessels used in the manufacture of therapeutic protein, whereby the container made of flexible film forms flexible disposable liner for the vessel. The disposable liner is formed separately from the rigid support and therefore can be placed on or into the rigid support for carrying out the process of the present invention, and can be removed from the support upon completion of the process. This is in contrast to a permanent liner that is formed on and adhered to the inner surface of the vessel.

The container can be formed by heat sealing one or more sheets of film of the fluoropolymer together, depending on the size and configuration of the container. Heat sealing involves welding overlapping lengths of the film together by applying heat to the overlap. The welding is achieved by heating the overlapped surfaces, usually under pressure, such as by using a heated bar or hot air, impulse, induction, infrared laser or ultrasonic heating. The overlapping film surfaces are heated above the melting temperature of the fluoropolymer to obtain a fusion bond of the overlapping film surfaces. An example of heat sealing of overlapping films of FEP (melting temperature of about 260° C.) is as follows: A pair of hot bars are heated to 290° C. and pressed against overlapping FEP film having a total film thickness of 5 mils (0.125 mm) under a pressure for 30 psi to provide the fusion seal in 0.5 sec. For ETFE overlapping films, each 4 mils (0.1 mm) thick, the hot bars of the impulse sealer are heated to 230° C. under a pressure of 60 psi (42 MPa) for about 10 sec to obtain the fusion seal. Typically the heat sealing can be completed in no more than 15 sec. Lower temperatures can be used for lower melting fluoropolymers. Typically the heat sealing can be completed in no more than 5 sec. Additional information on heat sealing is provided in S. Ebnesajjad, Fluoroplastics, Vol. 2, Melt Processible Fluoropolymers, published by Plastics Design Library 2003, pp. 493-496. The ports of entry into and exit from the container can be welded to the film by heat sealing techniques or by the welding and sealing techniques applied to various fluoropolymers as disclosed on pp. 461-493 of Fluoroplastics.

After fabrication of the container, because of the flexibility of the film from which it is made, the container, which is also flexible, can be collapsed as if it were a bag. The film, preferably after fabrication into a container, can be sterilized by known means, such as exposure to superheated steam or dry hot air or such chemical treatment as hot hydrogen peroxide or ethylene oxide or radiation. Ionizing radiation is preferred and gamma or electron beam (e-beam) radiation is especially preferred because of the sterilization effectiveness of radiation and its avoidance of residual chemicals from the chemical treatment sterilization of the film (container) so as not to contaminate the manufacture of the protein with such chemical or its residue. Preferably, the bag is inserted into a sealable overwrap, which is sized to enable the bag to fit within the overwrap. Alternatively, the bag may be folded over upon itself, which enables a smaller size overwrap to be used. The overwrap itself is preferably flexible and therefore formed from a polymer film such as of about 1 to 10 mils (0.025 to 0.25 mm) in thickness. Since the overwrap is not used in the manufacture of the protein, it does not have to have the non-contaminating character of the fluoropolymer bag with respect to the manufacture of the protein. Inexpensive polymer films such as of polyolefin such as polyethylene or polypropylene, or polyester, such as polyethylene terephthalate can be used as the overwrap. The polymer film making up the overwrap can be formed into a bag of the size and shape desired by heat sealing using conditions suitable for the particular polymer being used. The same heat sealing can be used to seal the overwrap once the fluoropolymer bag is inserted into the overwrap.

Sterilization can then be advantageously carried out on the package resulting from the sealed overwrap containing the fluoropolymer bag, preferably by exposing the package to ionizing radiation, preferably gamma or e-beam radiation, in an effective dosage to achieve sterilization of the fluoropolymer bag. Typically, such dosage is in the range of about 25 to 40 kGy. AAMITIR 17-1997 discloses guidance for the qualification of polymeric materials that are to be sterilized by radiation, including certain fluoropolymers. By way of example, a bag made of two sheets of FEP film, each 5 mil (0.125 mm) thick, heat sealed together as described above on three sides to leave an open top and having a capacity of 5 liters is formed. Alternatively, the bag is made from two sheets of ETFE film, each 4 mils (0.1 mm) thick, heat sealed as described above. A bag of similar size of polyethylene terephthalate (PET) film 1.2 mil (0.03 mm) thick is also formed, and the FEP or ETFE bag is placed within the polyethylene terephthalate bag. The polyethylene terephthalate bag is heat sealed using an AudionVac-VMS 103 vacuum sealing machine operating on program 2 to heat seal the overlapping films of the PET bag with a 2.5 sec dwell time of a hot bar pressing the films together against an anvil. The machine first inflates the PET bag, followed by drawing a vacuum of 1 Bar on the interior of the bag, and then carrying out the heat sealing. The resultant vacuum sealed PET bag with the collapsed FEP or ETFE bag inside forms a flat package. The resultant package is exposed to gamma radiation from a C⁶⁰ source to provide a dosage of 26 kGy, which is a sufficient dosage to sterilize the FEP bag within the PET overwrap. The PET overwrap maintains the sterilized condition of the FEP bag until the PET overwrap is unsealed to make the bag available as a container for use in the process of the present invention. Terminal sterilization can also be carried out by exposing the package to steam.

A gusseted container is made by heat sealing flexible films of FEP or ETFE together at their edges. This container when filled with liquid medium has a rectangular shape when viewed from one direction and an upstanding elliptical shape when viewed in the perpendicular direction. Thus, the container when filled (expanded) has the shape of a pillow. This container can also be oriented in the horizontal direction so that a gusseted sidewall faces upward. The orientation of the container will determine where the ports (openings) are positioned. In the embodiment next described, the container is oriented vertically, so that the gusseted sidewalls are vertical. The gussets can be formed from separate pieces of film or can be formed integrally with the sidewall. For example, a heat-sealed film in the shape of a tube can be pinched to form inwardly extended pleats, which are heat sealed at their top and bottom to retain the pleat shape, when the container is collapsed. The bottom and top of the tube shape is heat sealed to form the container. When the container is expanded, the pleats unfold at their midsections, to form gussets in the side of the container. In a different embodiment, the elliptical-shaped gusset sidewalls of the container are made from FEP or ETFE film cut into this elliptical shape. The sidewall are heat sealed to the rectangular front and back walls of the container by impulse heating, which involves a controlled heat-up applied to overlapping film portions, clamped between a heat bar and an anvil, heat sealing of the clamped film portions together, and controlled cooling of the seal while still under clamping pressure. The heat bar and anvil are shaped to the configuration needed for the desired shape of the heat seal. When the container used as a bioreactor, three ports are provided at the top of the container spaced along the upper rectangular edge, one for ingredient addition into the interior of the container, on for venting gas, notably carbon dioxide, that develops during the bioreaction, and the third port providing entry for a mixing blade. Three ports are also provided at the bottom rectangular edge of the container, one for drainage of liquid contents of the container, and the other two for introduction of oxygen and nitrogen into the interior of the container. Except for the presence of the ports, the container is a closed vessel. Each port is formed from tubing that has a valve for opening and closing the tubing. The tubing is heat sealed by impulse heating to the film walls of the container, i.e. the tubing is sandwiched between films forming opposite sides of the container and sealed around and to the periphery of the tubing. Alternatively, the ports can be integral with a base having tapered ends and the base is heat sealed to the opposing films. The interior volume of this container is 200 liters. When inflated by the addition of liquid medium, the container can be supported within a rectangular tank, the bottom edge of the container resting on the bottom of the tank, which has an aperture through which the tubing of the three bottom ports can extend, and the elliptical sidewalls being supported by the corresponding sidewalls of the tank, and the rectangular sidewalls contacting the corresponding sidewalls of the tank to provide support. After fabrication of this container, the flexibility of the FEP film enables the container to collapse into a flat shape, which can be heat sealed into an overwrap and then sterilized by exposure of the resultant sealed package to gamma radiation as described in the preceding paragraph. The gamma radiation also sterilizes the ports heat sealed into the container.

Such gusseted container is also applicable to other steps in the protein manufacturing process, except that when used in other steps, a different number of ports may be heat sealed to the container, depending on the needs of the process step. For example, ports for introducing or withdrawing gas will generally be unnecessary in other process steps. In any event, when the container is replaced, the ports integral with the container are also replaced with the sterilized container replacement. The gusseted container when used for storage of highly purified water (WFI) may simply have entry and exit ports or simply a single port that serves both purposes.

Details of the testing for extraction of organics from polymer film containers that had been subjected to 40 kGy gamma radiation are as follows:

The container of flexible film is filled with 250 ml of WFI or other test liquid and the resultant filled container is heated at 40° C. for 63 days. During this time, the corrosive WFI or other test solution has the opportunity to extract organics (organic compounds) from the film from which the container is made. Whether this extraction occurs or the extent of its occurrence is determined by subjecting samples of the WFI or other test liquid, as the case may be, to separation by gas chromatography, followed by analysis of the separation products by detection means. Volatile organic compounds (VOC) extracted in this process and separated in an HP 6890 GC (column: SPB-1sulfur, 30 m×0.32 mm ID, 4.0 micrometer thick film, operating at a range of 50-180° C.) are determined using a flame ionization detector (FID). The sample of test liquid is injected into the column at a temperature at 270° C. The flame detection pattern is electronically compared to a library of patterns in order to identify the organic present in the WFI or other test liquid. The separation of individual VOCs is based on retention time in the column, and the identification of the VOCs is done by their ionization signature.

Higher molecular weight organics that might be extracted from the stored, heated container can be considered as semi-volatile organic compounds (semi-VOC) and are also subjected to separation in a GC column, followed by detection of any semi VOC present. The column used for separating samples of the WFI or other test liquid is a GC (HP 6890) column, 30 m×250 micrometer ID, utilizing a 0.25 micrometer HP-5MS film, and the separated sample passing through the column is analyzed by Mass Spectrometer (MS) analysis using an HP 5973 MSD analyzer. The sample is injected into the column at 220° C. For the semi-VOC analysis the sample of WFI or other test liquid is spiked with 1000 ppb of 2-fluorobiphenyl (internal detection standard) and extracted several times with methylene chloride. The VOCs and semi-VOCs form a boiling point continuum of the organics that might be extracted from the container of polymer film being tested. The limits of detection of the VOCs and semi-VOCs are 50 ppb. The reporting of zero (0) for detection of extractables from the WFI and other test liquids in Tables 1 and 2 means that if extractables were present, they were present in less than 50 ppb.

The heated storage conditions for the WFI or other test liquid in the flexible film container, together with the GC separation of any organics present in a sample of the test liquid after such storage in the container, and analysis of the GC effluent can simply be referred to herein as the extraction test (long term).

As stated above, with respect to the containers of flexible film of fluoropolymer and containing WFI, no organics were detected in the extraction test.

The results of subjecting bags of fluoropolymer film and of film of different polymers to WFI and to other extractants to the extraction test are shown in Tables 1 and 2.

TABLE 1
Comparison of Extraction test results - Semi-VOC

| | Organics in Extraction Liquid | | | |
|---|---|---|---|---|
| | After Extraction (ppb) | | | |
| Extractant liquid | FEP | ETFE | EVA* | PE** |
| WFI | 0 | 0 | 0 | 570 |
| 1N HCl, 15 wt % NaCl | 0 | 0 | 0 | 0 |
| 1N NaOH, 15 wt % NaCl | 0 | 0 | 74 | 70 |
| PBS (phosphate buffered saline) | 0 | 0 | 0 | 210 |
| Guanidine HCl | 0 | 0 | 0 | 395 |

*laminate in which ethylene/vinyl acetate copolymer is the interior layer
**laminate in which ultra low density polyethylene is the interior layer.

TABLE 2
Comparison of Extraction test results - VOC

| | Organics in Extraction Liquid | | | |
|---|---|---|---|---|
| | After Extraction (ppb) | | | |
| Extractant liquid | FEP | ETFE | EVA | PE |
| WFI | 0 | 0 | 1395 | 2946 |
| I N HCl, 15 wt % NaCl | 0 | 0 | 2820 | 2961 |
| 1 N NaOH, 15 wt % NaCl | 0 | 0 | 1247 | 1997 |
| PBS (phosphate buffered saline) | 0 | 0 | 1271 | 1820 |
| Guanidine HCl | 0 | 0 | 1127 | 1200 |

These extractants (challenge solutions) shown in Tables 1 and 2 mimic liquids that may be included in the biological material by virtue of the process for producing the biologic material, such as in the manufacture of cellular product such as therapeutic protein. As shown in these Tables, the bags made of either FEP film or ETFE film were far superior to the bags made from the indicated hydrocarbon polymers as the interior layer of the bag, i.e. the hydrocarbon contact layers were much more contaminating of the various extraction test liquids. The organics detected in the extraction liquids in the EVA and/or PE bags included the following: ethanol, isopropanol, and dimethyl benzenedicarboxylic acid ester. It is surprising that the FEP and ETFE films do not yield extractables, because the effect of the gamma radiation on these polymers is to cause degradation, by polymer chain scission, this effect being more severe for the FEP than the ETFE as shown by the physical test results in Tables 3 and 4. The degradation/crosslinking effect of gamma radiation on various fluoropolymers is discussed in Y. Rosenberg et al., “Low Dose γ Irradiation of Some Fluoropolymers; Effect of Polymer Structure”, J. Applied Science, 45, John Wiley & Sons, 783-795.

The variability of the extraction results with the hydrocarbon polymer bags, i.e. different challenge liquids give different extraction results for the same bag, is a cause of concern for the user because the extraction results with still different reagents, as may be encountered in use, are unpredictable. In contrast, the consistently low extraction values for the fluoropolymers gives confidence that this will extend to different reagents.

Another test was conducted in which samples of the bags mentioned above were exposed to desorption conditions in a clean stainless steel tube of a Perkin-Elmer ADT-400. The tube was heated at 50° C. for 30 min to generate volatiles from the bag sample. The resultant gases were then subjected to GC separation (HP 6890 GC) at a column temperature of 40° C. to 280° C. and calibrated with n-decane, and mass spectrometer analysis (HP 5973 MS detector). This is the outgas test. The detection limit is 1 ppm (1 microgram/gm). No outgas was detected for either the FEP film or the ETFE film. For the PE film, 67 ppm of organics were detected, which included isopropyl alcohol, branched alkane hydrocarbons, octane, alkene hydrocarbons, decane, dodecane, alkyl benzenes, 2,6-di-tert-butyl benzoquinone, 1,4-benzenedicarboxylic acid, dimethyl ester, and 2,4-bis(1,1-dimethylethyl)-phenol. For the EVA film, 140 ppm of organics were detected, which included acetic acid, heptane, octane, branch alkane hydrocarbon, octamethyl cyclotetrasiloxane, decamethyl cyclopentasiloxane, alkyl benzenepolysiloxane, alkyl phenol, and 2,6-di-tert-butyl benzoquinone.

The container made from film of either tetrafluoroethylene/-hexafluoropropylene copolymer or ethylene/tetrafluoroethylene copolymer exhibits far superior stability under exposure to conditions of extraction and outgasing.

The effect of gamma radiation on physical properties of several fluoropolymers was tested. Tensile strength and elongation was tested on extruded films 4 to 5 mil (102-127 micrometers) thick in accordance with ASTM D 638, before and after exposure to 40 kGy gamma radiation, with the results being as reported in Table 3.

TABLE 3
Tensile Strength and Elongation of Fluoropolymer films

| | ETFE | PVDF | FEP |
|---|---|---|---|
| Tensile strength, psi (MPa) | | | |
| Before radiation | (62.3)8900 | (56)8000 | (42.7)6100 |
| After radiation | (52.5)7500 | (56.7)8100 | (26.6)3800 |
| Elongation at break, % | | | |
| Before radiation | 430 | 310 | 460 |
| After radiation | 440 | 140 | 450 |

These results show that the radiation greatly weakens the PVDF (polyvinylidenefluoride) and FEP (tetrafluoroethylene/hexafluoropropylene copolymer), greatly reduced tensile strength in the case of FEP and greatly reduced elongation in the case of PVDF. The reduction in elongation for PVDF manifests itself as reduced flexibility for the film making up the container, making it prone to cracking upon flexing.

The effect of 40 kGy of gamma radiation on fluoroethylene (polytetrafluoroethylene) is even more severe than for the PVDF and FEP. Both tensile strength and elongation deteriorate to lower levels than for the PVDF and FEP.

The films forming the subject of testing, the results of which are shown in Table 3, were also subjected to tear resistance testing in accordance with ASTM D 1004-94a, wherein the test specimen has a notch stamped therein as shown in FIG. 1 of the ASTM test procedure. In this test, the test specimen is gripped between pairs of jaws and pulled apart at a rate of 51 mm/min, which concentrates the stress at the notch in the test specimen. As the jaws are pulled apart, a graph of load required vs. extension of the test specimen in the notch region is formed. The resultant curve is plotted until the load reaches a peak and then declines 25% from the peak or until the specimen breaks, whichever occurs first. The area under the curve as determined by the computer program MathCAD represents the energy required to break the film. This test simulates the localized stresses that might be imposed on the container made from the film, such as might be encountered by contact with a sharp object or development of internal pressure within the liquid contents of the container. High load accompanied by low elongation in the tear resistance test has the disadvantage that the film will tend to puncture rather than elongate when subjected to localized stress. Moderate load accompanied by high elongation provide greater resistance to puncture. Table 4 shows the energy to break for the films of Table 3.

TABLE 4
Energy to Break

| | Gamma Radiation | Energy at Break |
|---|---|---|
| Film | Dosage(KGy) | (cm · N/cm) |
| ETFE | 0 | 2250 |
| | 25 | 2695 |
| | 40 | 2694 |
| PVDF | 0 | 1205 |
| | 25 | 1033 |
| | 40 | 753 |
| FEP | 0 | 1085 |
| | 25 | 1819 |
| | 40 | 1567 |

These results were obtained at room temperature (15-20° C.) tear resistance testing, averaging 5 test films/radiation condition. The energy at break values are normalized to the thickness of the film being tested, which accounts for the “cm” in the denominator.

It is preferred in the present invention that the energy at break of the film after exposure to 40 kGy of gamma radiation is at least 90% of that of the film prior to the radiation exposure, more preferably is at least as great after the radiation exposure as before. Table 4 shows no loss in energy at break for the ETFE film, when exposed to gamma radiation and substantially greater energy at break than either the PVDF or the FEP.

These physical testing results show that the ethylene/tetrafluoroethylene copolymer film bag is preferred over bags made from either PVDF or FEP because of the gamma radiation sterilizability of the ethylene/tetrafluoroethylene copolymer bag without appreciable detriment to either extraction of volatile compounds or in physical properties significant to the utility of the bag. Thus, the FEP and PVDF films used to make the flexible container according to the present invention should preferably be sterilized by methods other than gamma radiation, e.g. by exposure to e-beam radiation or by exposure to steam. If gamma radiation were used to sterilize perfluoropolymer such as FEP or radiation-degraded hydrofluoropolymer such as PVDF, these fluoropolymers would preferably be in the bag to be sterilized as the interior surface (film) of a laminate, in which the outer layer(s) of the laminate would be essentially not degraded by the radiation. Examples outer layer polymers are those disclosed above for use as the overwrap of the terminally sterilized package.

1. Process for the production of therapeutic protein, comprising
(a) preparing nutrient medium for fermenting or culturing of cells to express said protein.
(b) fermenting or culturing of said cells in the presence of said nutrient medium to express said protein,
(c) preparing protein separation solution for isolating said protein,
(d) formulating the isolated protein, and
(e) storing the formulated protein,
at least three of the steps (a)-(e) being carried out in separate disposable containers made of flexible film, at least the interior of said surface of said containers being fluoropolymer.

2. The process of claim 1 wherein steps (a), (b), and (c) are carried in said disposable containers.

3. The process of claim 1 wherein all said steps (a)-(e) are carried out in said disposable containers.

4. The process of claim 1 wherein said containers is entirely made of said fluoropolymer.

5. The process of claim 1 wherein said fluoropolymer is hydrofluoropolymer

6. The process of claim 1 wherein said fluoropolymer is perfluoropolymer.

7. The process of claim 1 and providing each said containers as a package comprising a sealed overwrap containing said container, said container being sterilized within sealed overwrap, thereby retaining its sterilized condition until opening of said overwrap.

8. The process of claim 1 and sterilizing each said containers by exposing them to ionizing radiation through their respective said sealed overwrap.

9. The process of claim 1 and additionally, storing water for injection in a container made of flexible film, at least the interior surface of said container being fluoropolymer and supplying said water to at least one of the steps (a)-(d).

10. The process of claim 9 wherein said supplying is to at least steps (a) and (c).

11. Process for storing water for injection (WFI), comprising storing said water in a container made of flexible film, at least the interior surface of said container being fluoropolymer.

12. The process of claim 11 wherein said container is disposable.
